(19) United States
(12) Patent Application Publication
(10) Pub. No.: US 2026/0249292 A1
(43) Pub. Date: Aug. 27, 2026

(54) A MICROFLUIDIC CHIP AND METHOD FOR SYNTHESIS OF NANOPARTICLES

(71) Applicant: UNIVERSITY COLLEGE DUBLIN, Dublin (IE)

(72) Inventors: Nan ZHANG, Dublin (IE); Dongsheng LIU, Dublin (IE); Mingzhi YU, Dublin (IE); Pranay SHAH, Dublin (IE); Mattew ALLEN, Dublin (IE)

(21) Appl. No.: 19/162,842

(22) PCT Filed: Mar. 8, 2024
(86) PCT No.: PCT/EP2024/056268

§ 371 Date: Sep. 5, 2025

(30) Foreign Application Priority Data
Mar. 9, 2023 (GB) 2303464.8

(51) Int. Cl. B01L 3/00; A61K 9/51

(52) U.S. Cl. CPC B01L 3/502746; A61K 9/5192; B01L 3/502715; B01L 2200/0647; B01L 2300/0867; B01L 2300/088; B01L 2300/0883; B01L 2400/0487; B01L 2400/086

(57) ABSTRACT

A microfluidic chip (1,100) microfluidic chip (1,100) comprising at least two inlets (2,4,4a-4j) that meet at a junction (5,5a,5b) that is in fluid communication with a proximal end (3) of a channel (8,8a,8b,8c) thereof and an outlet (6) that is in fluid communication with a distal end (7) of the channel (8), wherein the channel (8,8a,8b,8c) comprises either a plurality of spaced-apart baffle structures (10) continuous with an inner wall (12) of the channel (8, 8a,8b,8c) or a least one aerofoil-shaped baffle structure (14), or a combination thereof, and forms a microchannel (9) in the channel (8,8a,8b,8c) adapted to accommodate a fluid.

### RELATED PATENT APPLICATIONS

This patent application is a 35 U.S.C. 371 national phase patent application of PCT/EP2024/056268 filed on Mar. 8, 2024, entitled “A MICROFLUIDIC CHIP AND METHOD FOR SYNTHESIS OF NANOPARTICLES,” naming Nan ZHANG et al. as inventors, which claims priority to GB Application No. 2303464.8 filed on Mar. 9, 2023, entitled “A microfluidic chip for synthesis of nanoparticles.” The entire content of the foregoing patent applications is incorporated herein by reference, including all text, tables and drawings.

### FIELD OF THE INVENTION

The invention relates to microfluidic chips for use in synthesising nanoparticles. Specifically, the microfluidic chips comprise an aerofoil-shaped or tilted rectangular baffle structure inside and outside the walls of a microfluidic channel in a microfluidic chip.

### BACKGROUND TO THE INVENTION

Drug delivery systems in forms of nanoparticles have a broad spectrum of applications in area of gene therapy, cell therapy, and vaccine development. Lipid nanoparticles (LNPs) or liposomes that consist of a lipid bilayer and have a hollow structure are the most widely used drug carriers for nanomedicines. It is possible to encapsulate a variety of active pharmaceutical ingredients (APIs) (e.g., small hydrophobic, hydrophilic and amphiphilic molecules, peptides, antibody, plasmid-DNA, mRNA, siRNA etc.) in the lipid bilayer membrane or to encapsulate them into the hollow structure. However, significant concerns remain on efficacy, safety, consistency, scale-up of manufacturing and stability when translating LNPs from formulation into clinical application. LNPs' properties, e.g., size, size distribution, charge, drug encapsulation, transfection efficiency etc., can significantly affect the biodistribution and pharmacokinetics of the drug to be delivered. In order to obtain LNPs with expected characteristics, microfluidics that exploit fluidic control to synthesise LNPs have grown in acceptability and applications in laboratory and larger scales due to offering a reproducible and robust manufacturing through the precise control of flowing conditions.

In a microfluidic system, a microfluidic chip that determines mixing patterns is critical to achieve precise control of mixing reagents by controlling reaction time, mass transportation, and heat transfer, etc., which directly affects the properties of synthesised nanoparticles. However, flow within microfluidic channels in the chip usually occurs at low flow rates under a laminar flow regime, which makes homogenous mixing difficult to accomplish. Additionally, the formation of nanoparticles based on self-assembly is a fast process, and fast and uniform mixing is essential to achieve the required particle size and polydispersity index (PDI). Moreover, scale up of nanoparticle production from chemistry validation, in vitro screening, animal trials, clinical trials, and good manufacturing practice (GMP) production requires volume of nanoparticles from microliter, millilitre, to tens of litres and hundreds of litres, where low, medium, and high flow rates are required. Thus, a microfluidic channel with fast and uniform mixing, and scale up capability is required for synthesis of qualified nanoparticles.

Increasing the contact area of the fluids by lengthening the channel for a good mixing performance result in additional fluidic resistance to the channel, while increasing contact time by slowing the flow rate leads to a decreased production quantity of the microfluidic system. It is not suitable for scale up.

Rapid, controlled, and homogenous mixing is prerequisite for obtaining homogenous nanoparticles. Therefore, there is a strong need in chip design that provide good control of nanoparticle synthesis with desired properties at a wide range of flow rates and lipid/polymer compositions and ratios for formulation, and scale up.

It is an object of the present invention to overcome at least one of the above-referenced problems.

### SUMMARY OF THE INVENTION

The subject of this application is the provision of microfluidic (MF) chips comprising a series of microchannels for fast mixing of lipids/polymers with payloads, e.g. DNAs, RNAs, peptide and other relevant small molecules, to synthesis nanoparticles for a variety of applications such as gene therapy, cell therapy, and other drug development. To achieve fast mixing of reactants with a payload for highly homogenous nanoparticles, a baffle structure is incorporated into the microchannel of the MF chip. A tilted rectangular (an example of a non-aerofoil-shaped) baffle structure can be situated to the outer profile of the microchannel and/or an aerofoil-shaped baffle can be situated in the microchannels themselves (see FIGS. 2(c) and 5(d), for example). The non-aerofoil-shaped baffle structures (for example, a tilted rectangular baffle structure) are situated in the microchannels from the inlets to the junction where fluids from different inlets meet, while the aerofoil-shaped baffle structures are situated in the microchannels from the junction to the final outlet. A non-aerofoil baffle structure can create large pressure differences if flow is along or against a longitudinal direction of the channel, thus working as a back flow preventing structure (see FIGS. 8(a), 8(b), 17(a), and 17(b) for example). On the other hand, the non-aerofoil-shaped baffle structures significantly increase the flow resistance of channel as the flowrate increases compared to that without a baffle structure, thus creating larger pressure differences at inlets under different flow rates, which benefits to the control of flow rates driven by pressure. The microchannel with the aerofoil-shaped baffle structure can synthesis nanoparticles at a broad range of flow rates, and/or can mix different lipids, polymers with payloads. Aerofoil-shaped baffles can induce flow streams to split-and-recombine and/or fold-and-stretch the mixing solutions to enhance mixing performance, as well as localised vortices. A tilted rectangular (non-aerofoil-shaped) baffle can create large pressure differences if flow is along or against a longitudinal direction of the channel, thus working as a back flow-preventing structure. Both baffle arrangements can induce localized vortexes for homogeneous mixing.

In one aspect, there is provided a microfluidic chip as set out in the claims.

In one aspect, there is provided a system for synthesising nanoparticles as set out in the claims.

In one aspect, there is provided microfluidic chip (1,100) comprising at least two inlets (2,4,4a-4j) in fluid communication with at least one channel (205,405) that meet at a junction (5,5a,5b) that is in fluid communication with a proximal end (3) of a channel (8,8a,8b,8c) thereof and an outlet (6) that is in fluid communication with a distal end (7) of the channel (8,8a,8b,8c), wherein the channel (8,8a,8b,8c) comprise a plurality of spaced-apart, non-aerofoil-shaped baffle structures (10) continuous with an outer wall (11) of the channel (8,8a,8b,8c) and at least one aerofoil-shaped baffle structure (14) within the channel (8,8a,8b,8c), and a microchannel (9) is formed around the spaced-apart non-aerofoil-shaped baffle structures (10) and the at least one aerofoil-shaped baffle structure (14) in the channel (8,8a,8b,8c) adapted to accommodate a fluid. The at least one channel (205,405) may further comprise a plurality of spaced-apart non-aerofoil-shaped baffle structures (10) continuous with an outer wall (11) of the channel (205,405). The channel (205,405) is a backflow-suppressing channel and the non-aerofoil-shaped baffle structure (10) have a tilted rectangular baffle shape

In one aspect, the non-aerofoil-shaped baffle structure (10) has an angle (13) of about 40° to about 80° relative to the direction the non-aerofoil-shaped baffle structure (10) comes out from the inner wall (12) and the direction the fluid is flowing in the microchannel (9) or the channel (8,8a,8b,8c). The angle of the non-aerofoil baffle structure prevents the backflow of the fluid when pressure is applied to the MF chip.

In one aspect, the aerofoil-shaped baffle structure (14) comprises an upper surface (25), a lower surface (27), an angle of attack (23), a chord line (26), a leading edge (22), and a trailing edge (24). Preferably, the angle of attack (23) of the aerofoil-shaped baffle structure (14) is set at between about −120° to about 0° relative to the flow of the fluid.

In one aspect, the plurality of spaced-apart, non-aerofoil-shaped baffle structures (10) are distributed throughout the channel (8,8a,8b,8c).

In one aspect, the aerofoil-shaped baffle structure (14) is positioned between the spaced-apart non-aerofoil-shaped baffle structures (10) in the channel (8,8a,8b,8c).

In one aspect, the leading edge (22) of the aerofoil-shaped baffle structure (14) faces the direction of flow of the fluid in the channel (8,8a,8b,8c).

In one aspect, the channel (8,8a,8b,8c) has a formation selected from straight, a zig-zag, a circle, an oval, an ellipse, and any other curved shapes.

In one aspect, the channel (8a) is a lipid mixing channel (8a) positioned between, and in fluid communication with, the inlet (4,4a-4j) and the junction (5,5a,5b). Preferably, the channel (8a,8c) is a lipid nanoparticle synthesis channel (8,8c) in fluid communication with the junction (5) and the outlet (6). Preferably, the nanoparticle synthesising channel (8,8c) comprises the aerofoil-shaped baffle structure (14).

In one aspect, the aerofoil-shaped baffle structure (14) has a shape selected from a flat-convex, a wing, a sail, and a blade of a propeller, a rotor, or a turbine.

In one aspect, there is a microfluidic chip (1,100) comprising at least two inlets (2,4,4a-4j) in fluid communication with at least one channel (205,405) that meet at a junction (5,5a,5b) that is in fluid communication with a proximal end (3) of a channel (8,8a,8b,8c) thereof and an outlet (6) that is in fluid communication with a distal end (7) of the channel (8,8a,8b,8c), wherein the channel (8,8a,8b,8c) comprise an outer wall (11), an inner wall (12), and either a plurality of spaced-apart non-aerofoil-shaped baffle structures (10) continuous with the outer wall (11) of the channel (8, 8a,8b,8c) or a least one aerofoil-shaped baffle structure (14) that is continuous with the inner wall (12) of the channel (8,8a,8b,8c), and wherein a microchannel (9) is formed around the spaced-apart non-aerofoil-shaped baffle structures (10) or the at least one aerofoil-shaped baffle structure (14) in the channel (8,8a,8b,8c) adapted to accommodate a fluid.

In one aspect, the non-aerofoil-shaped baffle structure (10) has an angle (13) of about 40° to about 80° relative to the direction the non-aerofoil-shaped baffle structure (10) comes out from the inner wall (12) and the direction the fluid is flowing in the microchannel (9) or channel (8,8a,8b,8c).

In one aspect, the aerofoil-shaped baffle structure (14) comprises an upper surface (25), a lower surface (27), an angle of attack (23), a chord line (26), a leading edge (22), and a trailing edge (24). Preferably, the angle of attack (23) of the aerofoil-shaped baffle structure (14) is set at between about −120° to about 0° relative to the flow of the fluid.

In one aspect, the plurality of spaced-apart non-aerofoil-shaped baffle structures (10) are distributed throughout the microchannel (9) or channel (8,8a,8b,8c).

In one aspect, the aerofoil-shaped non-aerofoil-shaped baffle structure (14) is positioned between the spaced-apart baffle structures (10) in the microchannel (9) or channel (8,8a,8b,8c).

In one aspect, the leading edge (22) of the aerofoil-shaped baffle structure (14) faces the direction of flow of the fluid in the channel (8,8a,8b,8c).

In one aspect, the channel (8,8a,8b,8c) has a formation selected from straight, a zig-zag, a circle, an oval, an ellipse, and any other curved shapes.

In one aspect, the channel (205,405) is a backflow-suppressing channel (205,405) comprising the non-aerofoil-shaped baffle structure (10) having a tilted rectangular baffle shape.

In one aspect, the microfluidic chip (1,100) further comprises a lipid mixing channel (8a) positioned between, and in fluid communication with, the inlet (4,4a-4j) and the junction (5,5a,5b). The microfluidic chip (1,100) may further comprise a lipid nanoparticle synthesis channel (8c) in fluid communication with the junction (5) and the outlet (6). Preferably, the nanoparticle synthesising channel (8c) comprises the aerofoil-shaped baffle structure (14).

In one aspect, the microchannel (9) has a cross-sectional shape selected from a rectangle, a square, a circle, an oval, and an ellipse.

In one aspect, the width (16) of the microchannel (9) is from about 50 μm to about 1000 μm, while the height (18) of the microchannel (9) is from about 50 μm to about 500 μm.

In one aspect, the fluid has a total flow rate (TFR) of between about 100 μl/min to about 20 ml/min.

In one aspect, the microfluidic chip (1) is composed of a polymer selected from a cyclic olefine copolymer/homopolymer, poly (methyl methacrylate), polycarbonate-acrylonitrile butadiene styrene, polycarbonate, polyethylene terephthalate, polyethylene, polystyrene, polyimide, a polydimethylsiloxane, or a combination thereof.

In one aspect, there is provided a method for synthesising a nanoparticle, the method comprising the steps of: (a) applying an organic solution to at least one inlet (4,4c-4h) of the microfluidic chip (100) described above; (b) applying an aqueous solution to at least one inlet (2,4a,4b, 4i, 4j) of the microfluidic chip (100) described above that was not used in step (a); (c) applying a payload solution to at least one inlet (4b,4i) of the microfluidic chip (100) described above; (d) applying pressure to the inlets (2,4,4a-4j) to set a desired flow rate through the channel (205,405) and thus (8,8a,8b,8c); (e) mixing the payload solution of step (c) with the aqueous solution in the at least one inlet (4a,4j) in the channel (8b); (f) mixing a lipid solution with the organic solution of step (a) in the channel (8a); and (g) retrieving the synthesised nanoparticle from the outlet 6 that is in fluid communication with the channel (8,8c).

In one aspect, there is provided a method for synthesising a nanoparticle, the method comprising the steps of: (a) applying an organic solution to at least one inlet (4) of the microfluidic chip (1,100) described above; (b) applying an aqueous solution to at least one inlet (2) of the microfluidic chip (1,100) described above that was not used in step (a); (c) applying pressure to the inlets (2,4) to set a desired flow rate through the channel (205,405,8) and a desired flow rate ratio (FRR) between inlet (2) and inlet (4); (d) mixing a lipid solution with the organic solution of step (a) in the channel (8); and (e) retrieving the synthesised nanoparticle from the outlet 6 that is in fluid communication with the channel (8). A pressure drop can be adjusted by adjusting the number of baffles along the channel (205,405) for a given flow rate. It is like a regulator of pressure.

In one aspect, the FRR between the inlet (2) and the inlet (4) is usually more than 1, for example, 2:1, 3:1, 5:1, 9:1.

In one aspect, the channel (205) is symmetrical along the line connecting the inlet (2) and the junction (5).

In one aspect, the number of non-aerofoil-shaped baffle structures in channel (405) is usually more than that in the channel (205).

In one aspect, there is provided a microfluidic (MF) chip comprising a tilted rectangle baffle structure with both back flow suppression and flow rate control functions. The height of the MF chips is from about 25 um to about 1000 um. For tilted baffle-containing MF chips, the width of the channel is from about 50 um to about 1000 um. The angle of a zig-zag mixing channel is from about 20 degrees to about 90 degrees. The length of the baffle structure is from about 25 um to about 875 um. The baffle angles are from about 40 degrees to about 90 degrees, or preferably between about 40° and 80°. Baffle to baffle distance is from about 50 um to about 875 um.

In one aspect, the inlet has a volume of less than 1 ml, 900 μl, 800 μl, 700 μl, 600 μl, 500 μl, 400 μl, 300 μl, 200 μl, 100 μl, 50 μl, 10 μl, 5 μl, or 1 μl. Suitably, the inlet has a volume of from 1-1000 μl, 50-1000 μl, 50-900 μl, 50-800 μl, 50-700 μl, 50-600 μl, 50-500 μl, 50-400 μl, 50-300 μl, 50-200 μl. In one embodiment, the inlet has a volume of from 0.1-100 μl, 0.1-10 μl, or 0.1-100 μl. Preferably, the inlet has a volume of from 100-1000 μl, 100-900 μl, 100-800 μl, 100-700 μl, 100-600 μl, 100-500 μl, 100-400 μl, 100-300 μl, and 100-200 μl.

In one aspect, the channel has different total flow rate from 100 μl/min to 20,000 μl/min. The channels with baffle structures can synthesis nanoparticles at low total flow rate less than 1,200 μl/min, 1,100 μl/min, 1,000 μl/min, 900 μl/min, 800 μl/min, 700 μl/min, 600 μl/min, 500 μl/min, 400 μl/min, 300 μl/min, 200 μl/min, 100 μl/min. The baffle structures also can be used to mix lipid/polymer with organic solvent and payload with buffer, and prevent backflow. The aerofoil-shaped structures can enhance synthesis of nanoparticles at high total flow rate, for example, a total flow rate equal to or higher than 1 ml/min, such as 1.5 ml/min, 2 ml/min, 3 ml/min, 4 ml/min, 5 ml/min, 6 ml/min, 7 ml/min, 8 ml/min, 9 ml/min, 10 ml/min, 11 ml/min, 12 ml/min, 13 ml/min, 14 ml/min, 15 ml/min, 16 ml/min, 17 ml/min, 18 ml/min, 19 ml/min, and 20 ml/min.

In one aspect, the aerofoil-shaped baffle comprises an angle of attack (AOA) that is negative, that is, from about −120° to about 0°, a leading edge that faces the flow of liquids in the channel, an upper surface that is above the leading edge, a lower surface that is below the leading edge, and a trailing edge that is distal the leading edge. When in situ, the aerofoil-shaped baffle splits the channel of the MF chip into two sub-channels, i.e., the upper channel is above aerofoil upper surface, and the lower channel is below aerofoil lower surface. The width of lower channel is as uniform as possible by adjusting the shape of the outside baffle and the size of the radius of the trailing edge of the aerofoil. The shape of upper channel first shrinks to the minimum then expands. The ratio of the width of the lower channel to the minimum width of the upper channel is in the range of 1-2. The total width of the channel without any baffle is 100 um-2000 um, and ratio of the width of the channel to the depth of the channel varies from about 0.5:1 to about 2:1. For ease of manufacture, microchannels are generally rectangular in cross section, however, they can take any shape that is desired by the user, for example, a square, a circle, an oval, an ellipse, a polygon, and the like.

In one aspect, there is provided an integrated microfluidic (MF) chip, the MF chip comprises a tilted baffle structure (typically rectangular in shape) in the channel connecting inlets and the junction where fluids from different inlets meet, which aid in backflow suppression and flow rate control, and an aerofoil-shaped baffle structure within the channel connecting the junction and an outlet which aid in solution mixing and nanoparticle synthesis. Backflow suppression and flow rate control by pressure is achieved by using a tilted baffle structure at low to high flow rates, while the aerofoil-shaped baffle structure provides high efficiency, uniform mixing, and nanoparticle synthesis with low, medium, and high flow rates.

In one aspect, there is provided a system for synthesising nanoparticles, the system comprising one inlet configured to contain a carrier chemical composition, and the other inlet contains payload materials with buffer solutions, e.g. small hydrophobic, hydrophilic and amphiphilic molecules, peptides, antibody, plasmid-DNA, mRNA, siRNA etc. with tris buffer or phosphate-buffered saline (PBS) buffer etc. The system can also comprise up to 10 inlets, where each inlet can contain at least one chemical for carrier system. The example is the lipid nanoparticles containing four ingredients: ionizable lipid/cationic lipid, cholesterol, polyethylene glycol (PEG) lipid and a structural lipid. The channel's inlets for payload can be divided so that there is one for supplying buffer and one for supplying payload materials.

In one aspect, the small molecule is selected from an antibody, a nucleic acid, a peptide, a protein, and a hydrophilic or hydrophobic molecule, e.g. siRNA, mRNA, saRNA, tRNA, and the like.

In one aspect, the carrier is selected from a lipid, a polymer, or combinations thereof.

In one aspect, the channel has different total flow rates, ranging from 100 μl/min to 200 ml/min. The channels with tilted rectangular baffle structures can synthesis nanoparticles at low flow rates of less than 2,000 μl/min, for example, 1,900 μl/min, 1,800 μl/min, 1,700 μl/min, 1,600 μl/min, 1,500 μl/min, 1,400 μl/min, 1,300 μl/min, 1,200 μl/min. 1,100 μl/min, 1,000 μl/min, 900 μl/min, 800 μl/min, 700 μl/min, 600 μl/min, 500 μl/min, 400 μl/min, 300 μl/min, 200 μl/min, and 100 μl/min. The tilted rectangular baffle structures also can be used to mix lipids/polymers with buffer and payloads, and they can be also used to prevent backflow of liquid and liquid mixtures. The aerofoil-shaped baffle structures can synthesis nanoparticles at a high flow rate, for example, higher than 1 ml/min, such as 1.5 ml/min, 2 ml/min, 3 ml/min, 4 ml/min, 5 ml/min, 6 ml/min, 7 ml/min, 8 ml/min, 9 ml/min, 10 ml/min, 11 ml/min, 12 ml/min, 13 ml/min, 14 ml/min, 15 ml/min, 16 ml/min, 17 ml/min, 18 ml/min, 19 ml/min, 20 ml/min, 30 ml/min, 40 ml/min, 50 ml/min, 100 ml/min, and 200 ml/min.

In one aspect, the chip can be fabricated using polydimethylsiloxane soft lithography, plastic injection moulding using common plastic materials, including polycarbonate, poly (methyl methacrylate), cyclic olefine copolymer, cyclic olefine polymer, polystyrene, polylactic acid, polypropylene, polyethylene, and the like, and combinations thereof.

### Definitions

In the specification, the term “non-aerofoil-shaped baffle structure” should be understood to mean a body shaped which does not produce an aerodynamic reaction (lift) perpendicular to its direction of motion, for a small resistance (drag) force in that plane. The shape is typically a tilted rectangle, a triangle, a trapezoid or other polygon shape. The non-aerofoil-shaped baffle structure is continuous with the outer wall and protrudes inside the channel of the MF chip, forming microchannels between the non-aerofoil-shaped baffle structure and the opposite inner wall. The non-aerofoil-shaped baffle structure prevents backflow of the fluid with in the channel/microchannel of the MF chip. For example, a tilted rectangular (non-aerofoil-shaped) baffle can create large pressure differences if flow is along or against a longitudinal direction of the channel, thus working as a back flow-preventing structure. The non-aerofoil-shaped baffle arrangement can also induce localized vortexes for homogeneous (and enhanced) mixing.

In the specification, the term “aerofoil”, when used in relation to the aerofoil-shaped baffle of the claimed invention, should be understood to mean a body shaped to produce an aerodynamic reaction (lift) perpendicular to its direction of motion, for a small resistance (drag) force in that plane. The body shape can be, for example, a flat-convex shape (like a lens), a wing, a sail, or the blades of a propeller, a rotor, or a turbine. The upper surface of the aerofoil is generally associated with higher velocity and lower static pressure. The lower surface of the aerofoil has a comparatively higher static pressure than the upper surface. The pressure gradient between these two surfaces contributes to the lift force generated for a given aerofoil. The aerofoil-shaped baffle of the claimed invention improves the mixing of the fluid and particles inside the channel and microchannel by creating vortices that lend to homogenous mixing. The vortices induce flow streams to split-and-recombine and/or fold-and-stretch the mixing solutions to enhance mixing performance.

In specification, the term “baffle angle” should be understood to mean the angle between the longitudinal direction of a baffle structure situated to the outside walls of the microchannel and the forward flow direction of the liquid.

In the specification, the term “leading edge” should be understood to be the point at the front of the aerofoil-shaped baffle that has maximum curvature (minimum radius).

In the specification, the term “trailing edge” should be understood to mean the point of maximum curvature at the rear of the aerofoil-shaped baffle structure. The “chord line” is the straight line connecting the leading and trailing edges.

In the specification, the term “mean camber line” should be understood to mean the locus of points midway between the upper and lower surfaces. Its shape depends on the thickness distribution along the chord.

In the specification, the term “angle of attack” should be understood to mean the angle between a reference line on a body (often the chord line of the aerofoil structure) and the vector representing the relative motion between the body and the fluid through which it is moving.

In the specification, the term “split-and-recombine (SAR)” should be understood to mean a passive micro-mixing method that splits streams of liquid to be mixed into multiple smaller streams, and later rearranges them in alternating thin lamellae. It is typically characterized by the modification of channel geometries without external energy input and provides a simple manufacturing process, easy to implement, and has low energy consumption.

In the specification, the term “angle of attack (AOA)” should be understood to mean the angle between the chord line of an aerofoil-shaped baffle structure and the oncoming flow direction of the liquid.

In the specification, the term “a positive angle of attack (AOA)” should be understood to mean the oncoming flow of liquid hitting the upper surface of an aerofoil-shaped baffle structure, while the term “a negative AOA” should be understood to mean the oncoming flow of liquid is hitting the lower surface of an aerofoil-shaped baffle structure.

In the specification, the term “pressure drop” should be understood to mean the pressure difference between an inlet and an outlet of an MF chip.

In the specification, the term “total flow rate (TFR)” should be understood to the sum of flow rates of all inlets in the unit of volume/time, and is also equal to the flow rate through the mixing channel or outlet.

In the specification, the term “flow rate ratio (FFR)” should be understood the ratio of flow rate between aqueous solution and of organic solution from all inlets.

In the specification, the term “lipid” should be understood to mean a lipid selected from a triglyceride, glycerol, a diacylglycerol, a monacylglycerol, docosahexaenoic acid, dioleoylphosphatidylethanolamine (DOPE), 1,2-distearoyl-sn-glycero-3-phosphocholine (DSPC), dimethyldioctadecylammonium (DDAB), distearoyl-sn-glycero-3-phosphocholine (DSPC), 1,2-dimyristoyl-rac-glycerol-3-methoxypolyethylene glycol-2000 (DMG-PEG 2000), a medium-chain triglyceride, a lipase, an enzyme, a phospholipid, a sterol (such as cholesterol).

In the specification, the term “structural lipid” should be understood to mean a type of lipid that provides stability and structure to lipid nanoparticles (LNPs). In particular, they are the main component of the lipid bilayer that makes up the shell of LNPs. The most commonly used structural lipids in LNPs include phosphatidycholine (PC), cholesterol, and PEGylated lipids (PEG-lipids, such as 1,2-dimyristoyl-rac-glycerol-3-methoxypolyethylene glycol-2000 (DMG-PEG 2000)). PC and cholesterol are important for forming a stable and rigid lipid bilayer, while PEG-lipids are used to create a hydrophilic outer layer that helps the LNP avoid recognition by the immune system and prolong its circulation time in the bloodstream. These structural lipids also play a role in controlling the release of the encapsulated drug or genetic material from the LNP, by modulating the membrane permeability and stability.

In the specification, the term “structural lipid” should be understood to mean a type of lipid that provides stability and structure to lipid nanoparticles (LNPs). In particular, they are the main component of the lipid bilayer that makes up the shell of LNPs. The most commonly used structural lipids in LNPs include phosphatidycholine (PC), cholesterol, and PEGylated lipids (PEG-lipids, such as 1,2-dimyristoyl-rac-glycerol-3-methoxypolyethylene glycol-2000 (DMG-PEG 2000)). PC and cholesterol are important for forming a stable and rigid lipid bilayer, while PEG-lipids are used to create a hydrophilic outer layer that helps the LNP avoid recognition by the immune system and prolong its circulation time in the bloodstream. These structural lipids also play a role in controlling the release of the encapsulated drug or genetic material from the LNP, by modulating the membrane permeability and stability.

### BRIEF DESCRIPTION OF THE DRAWINGS

The invention will be more clearly understood from the following description of an embodiment thereof, given by way of example only, with reference to the accompanying drawings, in which:

FIG. 1 illustrates a perspective view of one embodiment of the MF chip with baffle structures of the claimed invention having a tilted rectangular shape, and where the total flow rate (TFR) of liquid is in the range of 100 μl/min to 1.2 ml/min.

FIG. 2(a) illustrates a plan view of the MF chip of FIG. 1 with tilted rectangular baffles in the outer walls of the MF channel, while FIG. 2(b) illustrates a partially enlarged plan view of FIG. 2(a) with tilted rectangular baffles in the outer walls. FIG. 2(c) illustrates a plan view of an MF chip with baffle structures of the claimed invention having a tilted rectangular shape and an aerofoil shape and where the TFR of liquid is in the range of 100 μl/min to 1.2 ml/min, as per FIG. 1; and FIG. 2(d) illustrates a perspective view of one embodiment of the MF chip with baffle structures of the claimed invention having a tilted rectangular shape and an aerofoil shape.

FIG. 3 illustrates the velocity streamline simulations of MF chip designs with baffle structures in the outer walls of the MF channel having a tilted rectangular shape (see the inset A).

FIG. 4 illustrates the concentration simulations of MF chip designs with tilted rectangular baffle structures in the outer walls of the MF channel.

FIG. 5 illustrates plan views of MF chip designs with (a) aerofoil-shaped baffle structures inside the MF channel; (b) with a zig-zag channel configuration, where the total flow rate of the liquid is in the range of 100 μl/min to 10 ml/min; while (c) shows a partially enlarged top view of chip designs with both non-aerofoil-shaped and aerofoil shaped baffle structures inside the MF channel; and (d)(i) shows a typical flat-convex aerofoil-shaped baffle structures, (d)(ii) a typical biconvex aerofoil-shaped baffle structure, and (d)(iii) a typical concave-convex aerofoil-shaped baffle structure, all of which can be used in the MF chip of the claimed invention.

FIG. 6 illustrates the velocity streamline simulations of the MF chip design of FIG. 2(c) and FIG. 5(b) at different total flow rates (a) 150 ul/min and (b) 3600 ul/min with flow split and recombine (SAR) and vortex formations, respectively.

FIG. 7 illustrates the concentration simulations of the MF chip design of FIG. 2(c) and FIG. 5(b) at different total flow rates (a) 150 ul/min and (b) 3600 ul/min with a FFR=3:1.

FIG. 8 illustrates a plan view of an MF chip design of the claimed invention with aerofoil-shaped and non-aerofoil-shaped baffle structures in the inside of the straight MF channel. The aerofoil-shaped baffle structures and MF channel can be amplified to adapt large total flow rates. It is a scaled-up version of the MF chips of (a) FIG. 5 and (b) FIG. 2(c), where the total flow rate of the liquid is in the range of 1 ml/min to 200 ml/min.

FIG. 9 illustrates the velocity streamline simulation of the MF chip of FIG. 8, where the total flow rate of the liquid is 4 ml/min. Inset B shows how flow split and recombine (SAR) and vortex formations are created in the flow around the aerofoil-shaped baffle structures.

FIG. 10 illustrates the pressure drop of the MF chip of FIG. 9 at a total flow rate of (a) 4 ml/min and (b) 20 ml/min.

FIG. 11 illustrates an integrated chip design combining aerofoil-shaped and tilted rectangular (non-aerofoil-shaped) baffle structures suitable for backflow-suppressing, lipid solution mixing, and lipid nanoparticle synthesis. The chip can comprise up to 10 inlets, where each inlet can contain at least one chemical, payload, or buffer for synthesis of nanoparticles.

FIG. 12 illustrates pressure plots (a) and (b) of backflow-suppressing channels at a flow rate of 2 ml/min using tilted rectangular baffle structures.

FIG. 13 illustrates concentration simulation (a)-(e) at a total flow rate of 4 ml/min with a flow rate ratio (between tris buffer with payload to ethanol with lipid (flow rate ratio between organic phase and aqueous phase) of 3:1. The tilted rectangular (non-aerofoil-shaped) baffle structures were used here for lipid mixing, while the aerofoil-shaped baffle structures were used for nanoparticle synthesis.

FIG. 14 is a bar chart illustrating the liquid nanoparticle size and polydispersity index (PDI) synthesised by the MF chip of FIGS. 2(a) and 2(c) at different flow conditions from 300 ul/min to 1200 ul/min.

FIG. 15 is a bar chart illustrating the liquid nanoparticle size and PDI synthesised by the MF chip of FIG. 8(a) (or FIG. 8(b)) at different flow conditions from 4.2 ml/min to 9 ml/min.

FIG. 16 shows the results of siRNA-LNPs synthesised by the MF chip of FIG. 2(c) under different siRNA concentrations at a total flow rate of 1.2 ml/min, where (a) is a bar chart illustrating the particle size and PDI synthesised, and (b) are images of A549 cell lines uptake results using siRNA-LNP.

FIG. 17 illustrates pressure plots (a) and (b) of backflow-suppressing channels at a TFR of 2 ml/min using tilted rectangular baffle structures.

### DETAILED DESCRIPTION OF THE DRAWINGS

### Materials and Methods

Acetone, 2-Propanol, Trichloro (1H,1H,2H,2H-perfluorooctyl) silane, Tris Buffer, Ethanol and Cholesterol are from Sigma-Aldrich (Darmstadt, Germany). Ultrasonic Cleaner is from GT Sonic. The polydimethylsiloxane (PDMS) surface was modified by oxygen plasma using a plasma cleaner (ZEPTO-W6, Diener electronic, Baden-Wurttemberg, Germany). Spin Coater was used to spread the adhesive voucher (Laurell Technologies, North Wales, United States). SU 8 2050 and SU 8 Developer are from Kayaku Advanced Materials (Massachusetts, United States). Hotplate is from SPS Polos (Germany). UV-KUB 2-UV-LED masking system is from KLOE (France). SYLGARD™ 184 Silicone Elastomer Kit (PDMS) is from Ellsworth Adhesives (Ireland). Microscope is from Brunel Microscopes (UK). Syringe pump is from KD Scientific Inc (United States). Syringe is from Becton, Dickinson and Company (New Jersey, United States). Litesizer 500 from Anton Paar (Graz, Austria) was used to measure nanoparticles size, PDI and Zeta-potential. Dimethyldioctadecylammonium (18:0 DDAB), 1,2-distearoyl-sn-glycero-3-phosphocholine (18:0 DSPC), 1,2-dimyristoyl-rac-glycero-3-methoxypolyethylene glycol-2000 (DMG-PEG 2000) are from avanti polar lipid Inc (Alabama, United States). Phosphate Buffered Saline Tablet is from Fisher Scientific (Pittsburgh, United States).

SU 8 2050 is used to fabricate master moulds using UV-lithography. Then, PDMS is poured onto the master moulds and is cured for a period of time, resulting in a PDMS MF chip of the claimed invention. Lipid mixtures were dissolved in ethanol at a 10:48:40:2 molar ratio for DSPC:Chol:DDAB:DMG-PEG 2000, respectively. TRIS buffer was added to inlet 1 using a syringe pump, and ethanol with the lipid solution was added to inlet 2, and the nanoparticle solution is collected from the outlet. DLS was used to measure lipid nanoparticles' size and PDI.

In order to realize the synthesis of nanoparticles with controllable properties, the present invention proposes a series of chip designs with baffle structures.

Microfluidic Chin Design for Low Flow Rate (Total Flow Rate Range: 100 μL/Min-1.2 mL/Min)

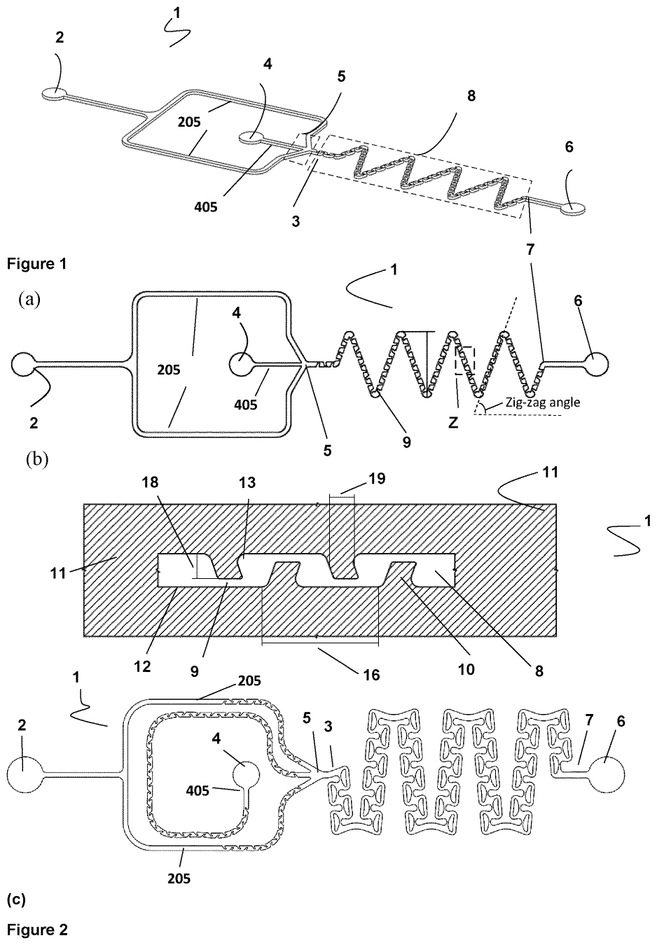

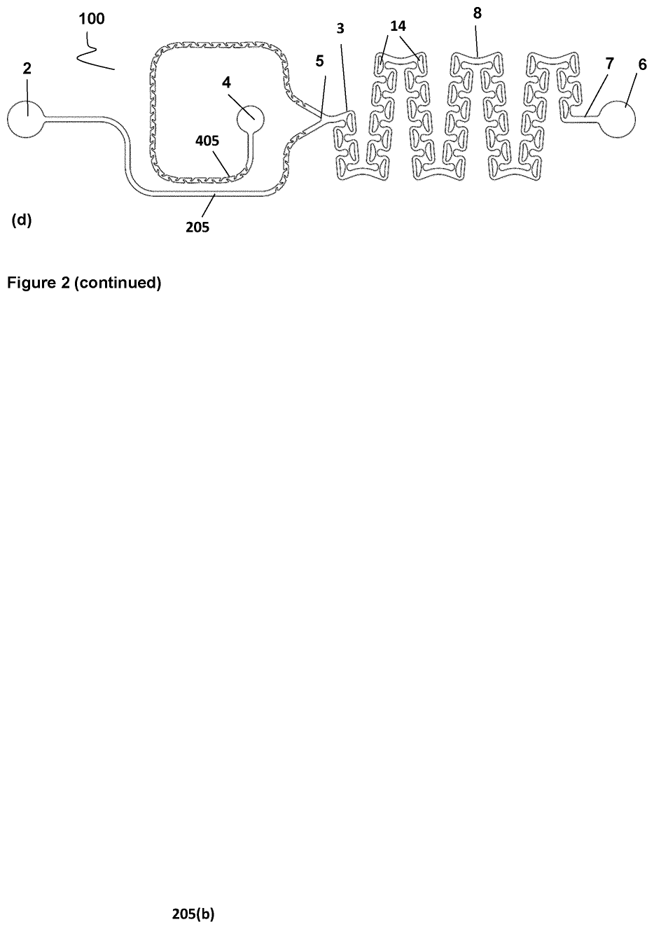

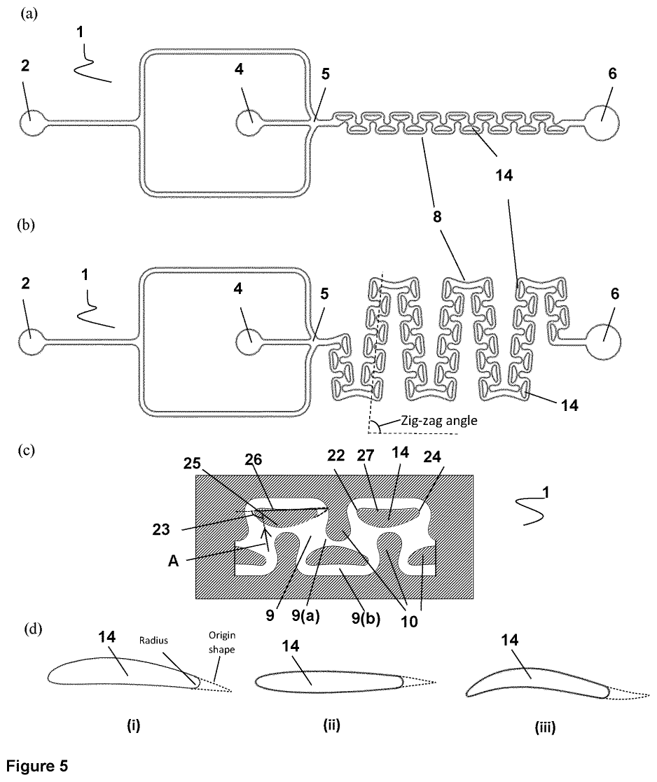

Referring now to the figures, where FIG. 1 illustrates a general embodiment of a microfluidic chip of the present invention. Specifically, FIG. 1 illustrates a perspective view of a microfluidic chip of the present invention, and is generally referred to by reference numeral 1. The microfluidic chip 1 shown in FIG. 1, FIG. 2(a), FIG. 2(c), and FIG. 2(d) comprises of at least two inlets 2,4 in fluid communication with each other at a junction 5, which itself is in fluid communication with at least one channel 205, 405, and a microchannel 9 (enlarged view seen in FIG. 2(b)) formed within the channels 205, 405. Each of the channels can be called a first channel 205, a second channel 405. A third channel 8 (8a,8b,8c) connects the junction 5 to an outlet 6, and the microchannel 9 is also formed within the channel 8 (see FIG. 2(b) and FIG. 5(b)). The microchannel 9 comprises an outer wall 11 and an inner wall 12. The outer wall 12 forming the outer boundary of the microchannel 9. In this aspect, the microchannel 9 further comprises a non-aerofoil-shaped baffle structure 10 that is continuous with the outer wall 11. The cross-section of the microchannel 9 shown here is rectangular, but it can be produced with whatever shape is desired. The microchannel 9 typically has a width 19 of from about 50 μm to about 1000 μm, a height 18 of from about 25 μm to about 500 μm, and an angle 13 where the non-aerofoil-shaped baffle structure 10 rises out from the inner wall 12 of from about 20° to about 90°, or from about 40° to about 80°. Two different reaction solutions (aqueous solution and organic solution) are added separately to the inlet 2 and the inlet 4, respectively. The organic solution contains carrier compositions, e.g., lipids, polymers, and the aqueous solution can contain payload materials with buffer solutions, e.g., small molecules, antibodies, nucleic acids, peptides, lipid nanoparticles, etc. with tris buffer or PBS buffer. When the solutions meet at the junction 5 and flow through the channel 8, the different solutions mix and react with each other as they flow through the microchannel 9. During this mixing and flowing through the microchannel 9 within the channel 8, nanoparticles are synthesised and finally exit via the outlet 6 and collected.

FIG. 2(a) is a plan view of FIG. 1, and shows the channel 8 having a zig-zag pattern, FIG. 2(b) shows an enlarged plan view of the insert Z of FIG. 2(a), while FIGS. 2(c),(d) show plan views of further embodiments of the MF chip 1 of the invention. The microchannel 9 is formed in the space created within the channel 8 between the protruding non-aerofoil-shaped baffle structure 10 and the inner wall 12 opposite therefrom. The microchannel 9 within the channel 8 typically has a meandering form, irrespective of the form of the channel 8, created by a plurality of, in this example, the non-aerofoil-shaped baffle structures 10 positioned within the channel 8 having a tilted rectangular form, and forming what is almost like a zig-zag pattern. In one aspect, rather than having a meandering or zig-zig form, the channel 8 can have a straight form or other form, wherein the microchannel 9 within the channel 8 retains its meandering form through the use of the non-aerofoil-shaped baffle structure 10.

To improve the synthesising performance of the microfluidic chip 1, the non-aerofoil-shaped baffles 10 are added to both sides of the outer walls 11 of the channel 8 in an alternating and evenly distributed pattern (see FIG. 2(a)). By adjusting the shape (e.g., height, width, angle) and the distribution of the non-aerofoil-shaped baffles 10, the geometry of the microchannel 9 can be changed (see FIG. 2(b)), thereby changing the liquid mixing mode, and improving the performance of the nanoparticle synthesis.

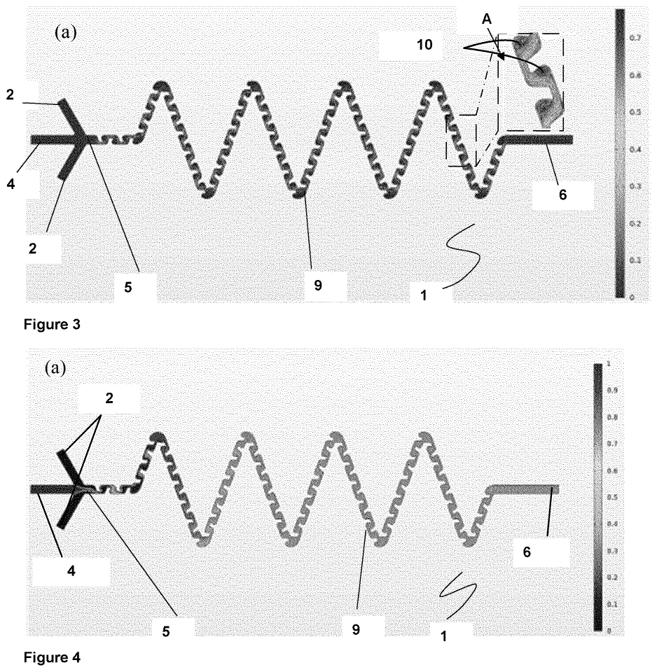

FIG. 3 shows a velocity streamline plot generated by the MF chip 1 of FIG. 2(a), when modelled by a computational fluid dynamics (CFD) simulation. The insert A of FIG. 3 show the microchannel 9 isolated from the channel 8 so that the vortexes generated by the flow of the solutions through the microchannel 9 are more evident. Local vortexes that promote the mixing performance of the solutions occur due to the presence of the non-aerofoil-shaped baffle structures 10 in the microchannel 9 (see the insert A of FIG. 3).

Mixing performance of the microfluidic chip 1 is verified by the concentration plots shown in FIG. 4, where the outlet concentration is uniform, one inlet 4 input concentration is 1, and the other inlet 2 is 0.

Microfluidic Chip 1 Design for Low and Intermediate Flow Rate (Total Flow Rate Range: 100 μL/Min~4 mL/Min)

FIG. 5 illustrates an MF chip 1 of the claimed invention where the microchannel 9 further comprises an aerofoil-shaped baffle structure 14 included with the non-aerofoil-shaped baffle structures 10. The aerofoil-shaped baffle structure 14 provides a fluid spilt-and-recombine (SAR) passive MF chip 1 which has increased mixing efficiency. The aerofoil-shaped baffle structure 14 typically has a surface shaped like an airplane wing, tail, or propeller blade. The aerofoil-shaped baffle structure 14 typically has a leading edge 22, a trailing edge 24, and a chord line 26. The aerofoil-shaped baffle structure 14 also comprises an angle of attack 23 (where the liquid meets the baffle 14 initially), an upper surface 25, and a lower surface 27 (see FIG. 5(c)). The aerofoil-shaped baffle structures 14 are modified by considering the radius of curvature of the trailing edge 24 to optimize manufacturability without impact mixing performance. In addition, the geometric characteristics of the aerofoil-shaped baffle structure 14 (the upper surface 25 of the structure 14 being more curved than the lower surface 27) causes the fluid flowing through the microchannel 9 to flow over the leading edge 22 at the angle of attack 23 of between about −120° to about 0° (see arrow A), and around the aerofoil-shaped baffle structure 14 upper surface 25 and lower surface 27 to generate a velocity difference at points 9(a) and 9(b), which further generates a pressure difference (a pressure drop). As a result, the flow of liquid through the microchannel 9 can fold and stretch at the trailing edge 22 of the aerofoil-shaped baffle structure 14 to promote mixing (see FIG. 5(c)). A vortex can be induced at a high flowrate, which further promotes the mixing of the fluid in the microchannel 9. FIGS. 1, 2(a), 2(b), 2(d), and 5(a) shows a channel 8 with a straight channel design and with flat-convex aerofoil-shaped baffle structures 14 (see FIG. 5(d)(i)) in the microchannel 9. FIGS. 2(c), 2(d), 5(b) shows the channel 8 with a zig-zag design, having more aerofoil-shaped baffle structures 14 in the microchannel 9. FIG. 5(c) further highlights the mixing of the non-aerofoil-shaped baffle structures 10 and aerofoil-shaped baffle structures 14 in the same channel 8. FIGS. 5(d)(i)-(iii) show three typical aerofoil-shaped structures 14.

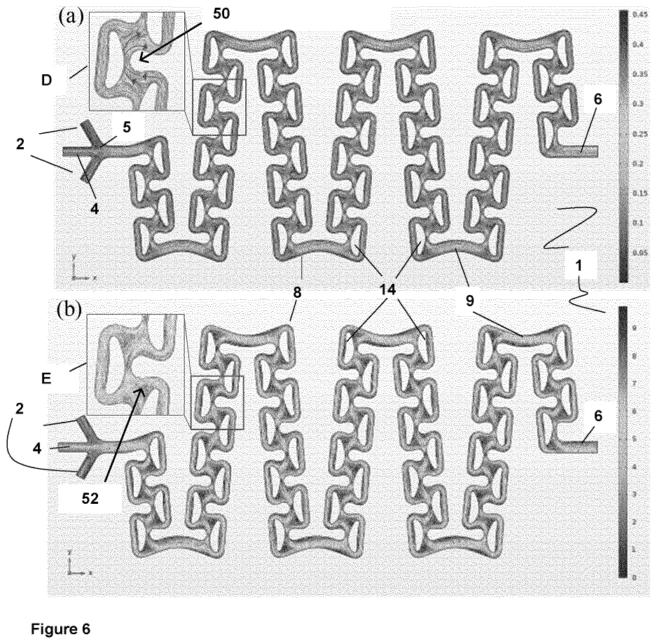

FIG. 6 (a) and FIG. 6(b) show velocity streamline plots generated by the microchannel 9 of the MF chip 1 of FIGS. 1, 2(a), 2(b), and 2(d) when modelled by a computational fluid dynamics (CFD) simulation at a low TFR of 150 μL/min and a high TFR of 3600 μL/min, respectively, where outlet concentration is uniform. Here, the phenomenon of split-and-recombine 50 (see the inset D) occurs at a low TFR of 150 μL/min, while a vortex 52 (see the inset E) occurs at a high TFR of 3600 μL/min.

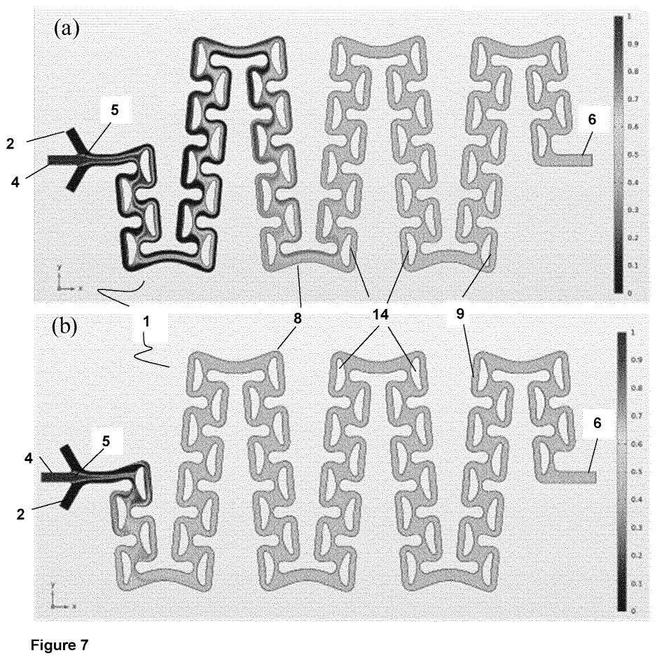

Mixing performance of the MF chip 1 is verified by the concentration plots shown in FIG. 7, where the outlet 6 concentration is uniform, one inlet 4 input concentration is 1, and the other inlet 2 is 0 (FFR=3:1). FIG. 7(a) and FIG. 7(b) show concentration plots at a low TFR of 150 μL/min and a high TFR of 3600 μL/min, respectively, where outlet concentration is uniform.

Microfluidic Chip 1 Design for High Flow Rate (Total Flow Rate Range: 2 mL/Min-200 mL/Min)

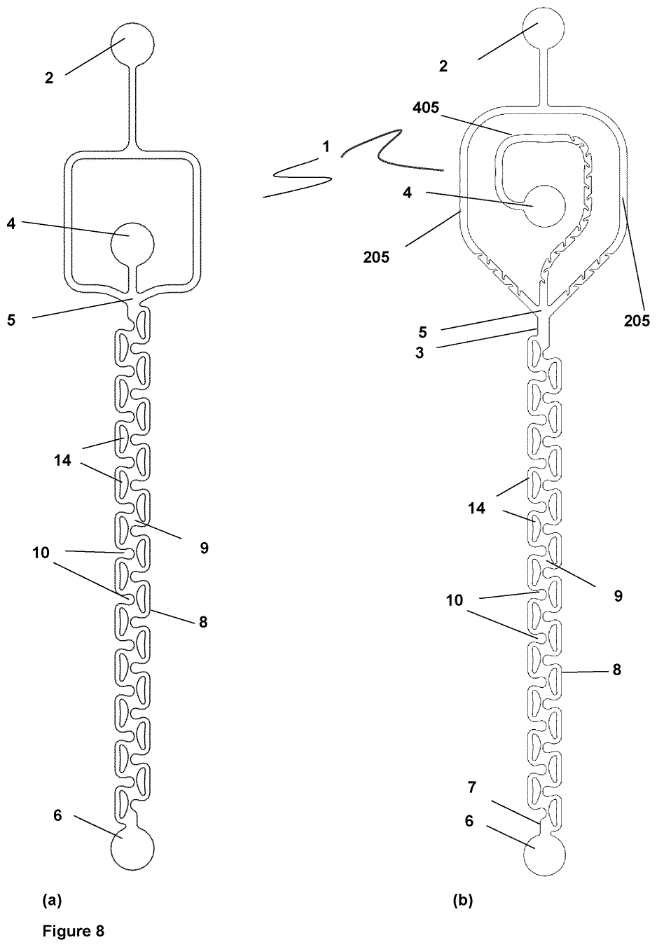

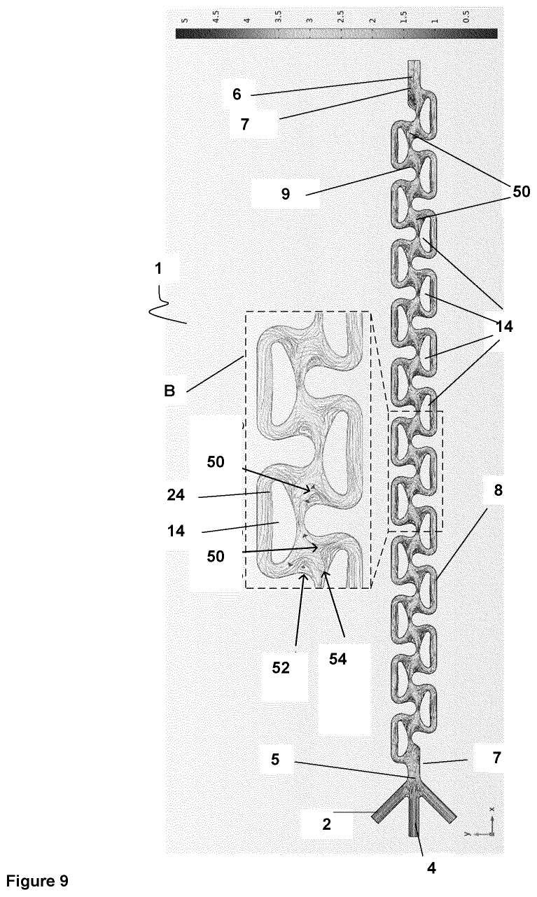

The MF chip 1 of the claimed invention can be scaled up for use with higher total flow rates (see FIG. 8(a),(b)). The MF chip 1 in FIG. 8(a) and FIG. 8(b) is the same as the MF chip 1 in FIGS. 5(a) and 2(c), respectively, magnified by a factor of 1.75. As the flow rate increases, the number of non-aerofoil baffle structures 10 within the channels 205405 and the number of aerofoil-shaped baffle structures 14 with the channel 8 should be less that of MF chip 1 shown in FIG. 2(c). The velocity streamline plot of the MF chip 1 in FIG. 8(a),(b) is at a total flow rate of 4 ml/min is shown in FIG. 9, where the flow of fluid folds and stretches 54 at the trailing edge 24 of the aerofoil-shaped baffle structure 14 in the microchannel 9 (see highlighted portion B). The vortex 52 phenomenon occurs in localized corners of the microchannel 9, while the split and recombine 50 phenomenon occurs around the path of flow of the fluid in the microchannel 9 where the fluid meets the junction where the non-aerofoil-shaped baffle structures 10 and the aerofoil-shaped baffle structures 14 are separated from each other. Both vortex and split-and-recombine can facilitate fluids stretching and folding to further improve the mixing quality.

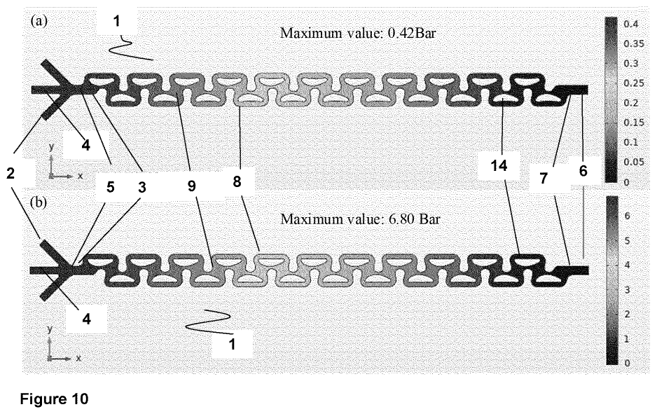

FIG. 10(a) and FIG. 10(b) illustrate plots of pressure drops at flow rates of 1.5 mL/min and 20 mL/min, respectively, where it is evident that the overall pressure drops. The pressure drop at a flow rate of 20 mL/min is 6.8 Bar, which is achievable by common microfluidic devices. The mixing channel 8 of the MF chip 1 in FIGS. 2(c)b and 5(a) can be magnified by a factor from 1 to 10 times to allow the flow rate increased from 20 mL/min to 200 mL/min by keeping low pressure drop.

### Integrated Microfluidic Chip Design

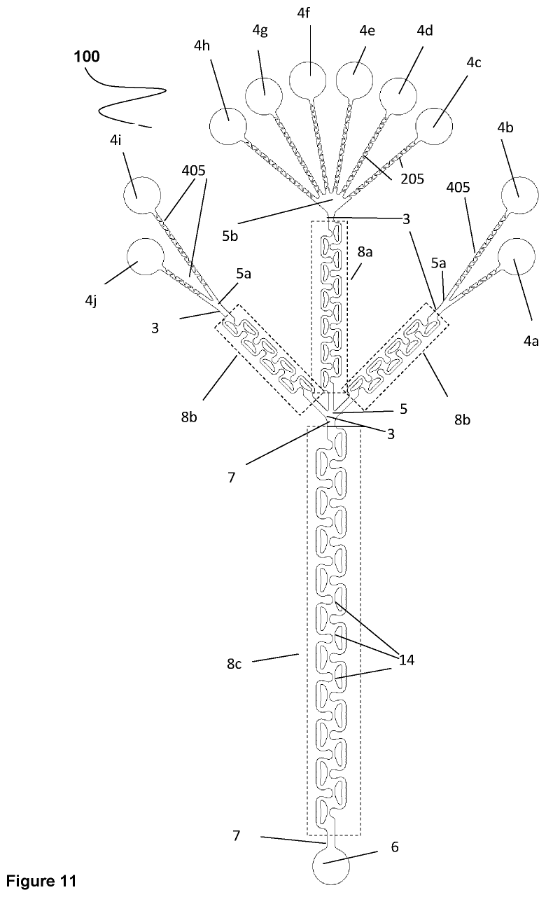

FIG. 11 presents an integrated MF chip 100 design by combining both the non-aerofoil-shaped baffle structure 10 having a tilted rectangular shape and the aerofoil-shaped baffle structure 14 in the channels 8. The MF chip 100 can comprise at least 10 inlets 4a-4j, and each inlet 4a-4j can contain at least one chemical composition of nanoparticle or organic solvent or buffer solution. The example illustrated here is for lipid nanoparticles containing four ingredients: an ionizable lipid/cationic lipid, cholesterol, polyethylene glycol (PEG), and a structural lipid, e.g., dioleoyl phosphatidylethanolamine (DOPE), 1,2-distearoyl-sn-glycero-3-phosphocholine (DSPC). The inlets 4c-4h can be utilised such that the one or more inlets can be used to deliver organic solutions. In the illustrated example, the inlets 4c,4h are used to deliver an ethanol solution, and the other inlets 4d,4e,4f,4g, are used to deliver dimethyldioctadecylammonium (DDAB), distearoyl-sn-glycero-3-phosphocholine (DSPC), dimyristoyl glycerol-Polyethylene glycol 2000 (DMG-PEG 2000), and cholesterol, respectively, at different flow rates. Buffer and payload solution inlets 4i,4j flow down to a junction 5a and first are mixed in a payload mixing channel 8b to produce a homogenous aqueous solution. Lipids and ethanol solutions from inlets 4c,4d,4e,4f,4g and 4h flow through a backflow-suppressing channel 8a and down to a junction 5b, and first are mixed in a lipid mixing channel 8a to produce a homogenous lipid solution. The backflow-suppressing channel 405 comprises the non-aerofoil-shaped baffle structure 10 having a tilted rectangular shape, which can induce a lower pressure drop for forward flow than backward flow. The backflow-suppressing channel 405 can reduce the time to stabilise flow of the components for lipid nanoparticle synthesis, resulting in a significant reduction in waste volume. Payload solution (for example, mRNA, siRNA, plasmid-DNA) flow through the backflow-suppressing channel 405 from inlets 4b,4i mix with buffer solutions from inlet 4a,4j to form homogenous buffer/payload solution at the lipid mixing channel 8b. The homogenous lipid solution leaves a lipid mixing channel 8a via the junction 5 where it meets with and mixes with the buffer and payload mixture from the channels 8b and progresses to nanoparticle synthesis channel 8c.

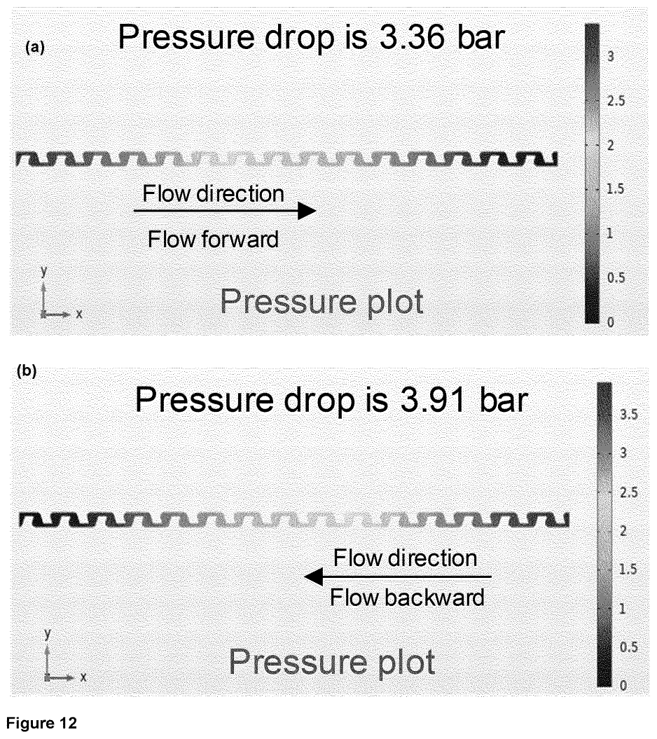

The backflow-suppression channel 8a can obviously stop the backflow due to a high flow resistance when flow is against flow direction. The CFD simulation results of the backflow-suppression channel 8a at a flow rate of 1.5 ml/min are shown in FIG. 12. It is obvious from the pressure plots that there a significant difference between the forward flow (3.36 Bar) and the backward flow (3.91 Bar, increased by 16.4%) in terms of pressure drop values. An increase of pressure by up to 40% can be achieved for backward flow using the backflow-suppression channel 8a. From the pressure plots, it can be seen that the backward flow has a larger flowing resistance than the forward flow. To achieve this, the angle of the non-aerofoil-shaped baffle structure 10 should be less than 90°, usually from 40° to 89°.

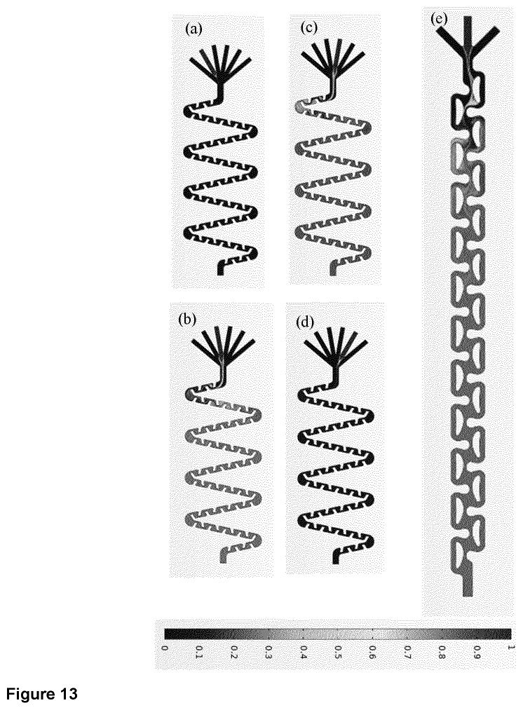

The results of lipid mixing in a MF chip 1 of the claimed invention with a total flow rate of 4 ml/min and a flow rate ratio (between tris buffer to ethanol with lipid (flow rate ratio between organic phase and aqueous phase) of 3:1, are illustrated in concentration plots shown in FIG. 13. Concentration plots of DDAB, DSPC, cholesterol, and DMG-PEG 2000 are illustrated in FIGS. 13(a)-(d), respectively, which occur in the lipid mixing channel 8c (see FIG. 11). FIG. 13(e) shows the concentration plot in the lipid nanoparticle synthesis channel 8d. It can be seen that good mixing performances are achieved from the homogenous concentrations of the fluid at the outlet 6.

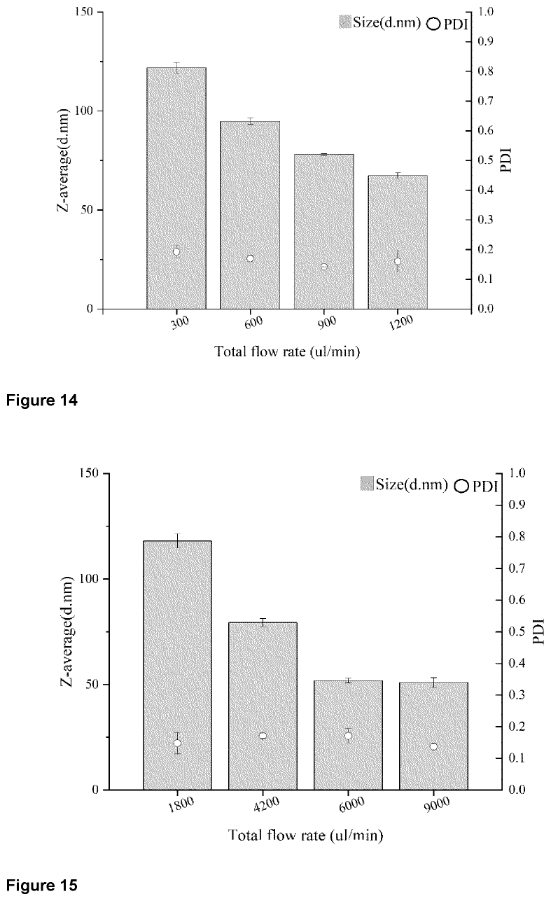

The designs of the MF chip 1,100 of the claimed invention were made using polydimethylsiloxane (PDMS) soft lithography. Four different lipids were used to synthesis lipid nanoparticles (LNPs): 18:0 dimethyldioctadecylammonium (DDAB), 18:0 1,2-distearoyl-sn-glycero-3-phosphocholine (DSPC), cholesterol and dimyristoyl glycerol-Polyethylene glycol 2000 (DMG-PEG 2000). The molar ratio is 40:10:48:2. The synthesised LNPs were diluted at 20% in PBS buffer and then for size measurement. Litesizer™ 500 from Anton Paar was used for DLS measurement to obtain size and PDI. The synthesised nanoparticle size are from 45 nm to 125 nm with a low polydispersity index (PDI). The size results of synthesised nanoparticles are shown in FIGS. 14, and 15. Also, the LNP synthesis tests are conducted using the microfluidic chip 1 shown in FIGS. 2(c) and 5(b) and the enlarged MF chip 1 (magnified 1.75 times in size) as shown in FIG. 8(a), (b), where the nanoparticle size ranges from 120 nm to 50 nm with PDI lower than 0.2 with good uniformity (FIG. 14 and FIG. 15).

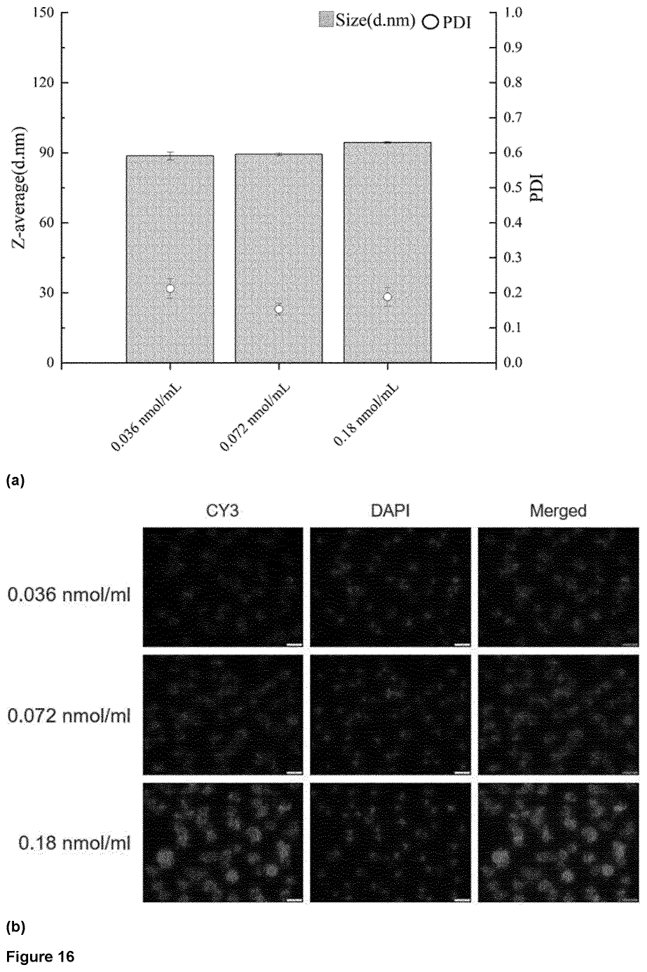

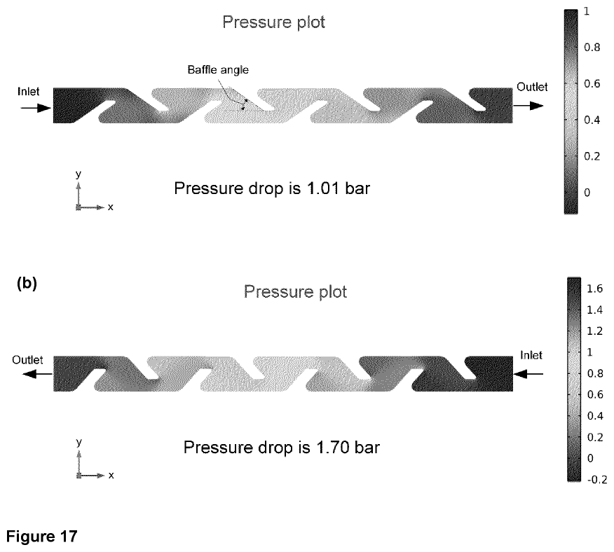

The results of lipid and siRNA (CY3 labelled) mixing in the MF chip 1,100 of the claimed invention with a total flow rate of 1.2 ml/min and a flow rate ratio (between tris buffer contain siRNA to ethanol with lipid) of 3:1, are illustrated in FIG. 16(a). Firstly, Tris buffer with different siRNA (CY3 labelled) concentration was connected with the inlet 2. The siRNA concentrations were 0.24, 0.48 and 1.2 nmol/ml respectively. Ethanol with 8 mg/ml lipid was connected with the inlet 4. The weight ratio between DDAB, DSPC, cholesterol and DMG-PEG 2000 was 40:10:48:2. After collecting siRNA-LNP solution from the outlet 6, siRNA-LNP solution was diluted at 20% in PBS buffer. The finial siRNA concentration was 0.036, 0.072 and 0.18 nmol/ml. Then, a light scattering analyser (Litersizer) was used to measure size and PDI. The average size of different groups are 88.64 nm, 89.37 nm and 94.34 nm, and PDIs are 0.212, 0.153 and 0.188, as shown in FIG. 16(a).

A549 cells with 5,000 cells per well in 200 ul complete Dulbecco's modified Eagle's medium (DMEM) were seeded in a 96-well plate in an incubator at 37° C. in a humidified atmosphere with 5% CO2 for 24 hours. Then, complete DMEM was replaced by DMEM and siRNA-LNP mixture solutions with a ratio of 150 ul DMEM and 50 ul siRNA-LNP. After incubating for 6 hours, mixture solutions were replaced with PBS with NucBlue Fixed Cell ReadyProbes Reagent. After incubating for 10 mins, the A549 uptake results were checked under an epifluorescence microscope, which are shown in FIG. 16(b). The uptake results demonstrate that A549 cells absorb a greater amount of siRNA as the concentration of siRNA increases.

### DISCUSSION

The MF chips 1,100 of the claimed invention achieve a flow rate from 100 μL/min to 200 mL/min, synthesising LNPs having acceptable nanoparticle size and PDI by using aerofoil-shaped structures 14 and/or the non-aerofoil-shaped baffle structures 10 having a tilted rectangular shape within the microchannel 9. The design of the MF chip 1 permits them to be scaled up. An example of the scaled-up MF chip 1 is illustrated in the MF chip 100 and which combines the aerofoil-shaped baffle structures 14 and the non-aerofoil-shaped baffle structures 10 having a tilted rectangular shape. The combination of the non-aerofoil-shaped baffle shapes 10 and the aerofoil-shaped baffle structures 14 allows individual control of mixing of the multiple chemical reactants and then synthesis of nanoparticles with payloads. The pressure required for a high flow rate is easily achieved using a syringe pump or other mechanical/pressure pumps. Some advantages are as follows:
(1) Rapid, controlled, and homogenous mixing is achieved by introducing a non-aerofoil-shaped baffle structure 10 and an aerofoil-shaped baffle structure 14 to the microfluidic mixing channels 8,8a,8b,8c, and synthetic production results are controllable by changing flow conditions and reactant chemicals ratios. The flow conditions include flow rate, aqueous:organic solution ratio, which influence the results of the outcome of the synthetic production.
(2) Mixing modes of fluid flow can be easily controlled by changing geometries of the non-aerofoil-shaped baffle structure 10 in the outer walls of the channel 8 and the aerofoil-shaped baffle structure 14 inside the channel 8.
(3) The microfluidic mixing channels 8,8a,8b,8c are optimized for controlling the size and PDI of synthesised nanoparticles over a wide flow rate, which ranges from 150 uL/min to 20 mL/min, and enables the reproducible scaled-up production.
(4) Channels 205, 405 suppress the backflow and reduce the time to stabilise flow rates for lipid nanoparticle synthesis, resulting in a significant reduction in waste volume.
(5) Controlling the individual flow of at least one reactant chemical from the inlets 4c,4d,4e,4f,4g,4h, and delivering the payload from inlet 4b,4i and the buffer from inlet 4a,4j, which allows mixing of various reactant chemicals together. The use of the non-aerofoil-shaped baffle structure 10 with the tilted rectangular shape provides a back flow suppression channel 405, and then continuing the synthesis of the nanoparticles in the mixing channel 8c with the aerofoil-shaped baffle structures 14. The combination of these elements means that various formulations of nanoparticles can be synthesized without fuss.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

All publications, patents, patent applications and other references mentioned herein are hereby incorporated by reference in their entireties for all purposes as if each individual publication, patent or patent application were specifically and individually indicated to be incorporated by reference and the content thereof recited in full.

1. A microfluidic chip (1,100) comprising at least two inlets (2,4,4a-4j) in fluid communication with at least one channel (205,405) that meet at a junction (5,5a,5b) that is in fluid communication with a proximal end (3) of a channel (8,8a,8b,8c) thereof and an outlet (6) that is in fluid communication with a distal end (7) of the channel (8,8a,8b,8c), wherein the channel (8,8a,8b,8c) comprise a plurality of spaced-apart non-aerofoil-shaped baffle structures (10) continuous with an outer wall (11) of the channel (8,8a,8b,8c) and at least one aerofoil-shaped baffle structure (14) within the channel (8,8a,8b,8c), and wherein a microchannel (9) is formed around the spaced-apart non-aerofoil-shaped baffle structures (10) and the at least one aerofoil-shaped baffle structure (14) in the channel (8,8a,8b,8c) adapted to accommodate a fluid.

2. The microfluidic chip (1,100) according to claim 1, wherein the non-aerofoil-shaped baffle structure (10) has an angle (13) of about 40° to about 80° relative to the flow direction, and the non-aerofoil-shaped baffle structure (10) comes out from the inner wall (12) and the direction the fluid is flowing in the microchannel (9) or the channel (8,8a,8b,8c).

3. The microfluidic chip (1,100) according to claim 1, wherein the aerofoil-shaped baffle structure (14) comprises an upper surface (25), a lower surface (27), an angle of attack (23), a chord line (26), a leading edge (22), and a trailing edge (24).

4. The microfluidic chip (1,100) according to claim 1, wherein the aerofoil-shaped baffle structure (14) comprises an upper surface (25), a lower surface (27), an angle of attack (23), a chord line (26), a leading edge (22), and a trailing edge (24), and wherein the angle of attack (23) of the aerofoil-shaped baffle structure (14) is set at between about −120° to about 0° relative to the flow of the fluid.

5. The microfluidic chip (1,100) according to claim 1, wherein the plurality of spaced-apart non-aerofoil-shaped baffle structures (10) are distributed throughout the channel (8,8a,8b,8c).

6. The microfluidic chip (1,100) according to claim 1, wherein the aerofoil-shaped baffle structure (14) is positioned between the spaced-apart non-aerofoil-shaped baffle structures (10) in the channel (8,8a,8b,8c).

7. The microfluidic chip (1,100) according to claim 1, wherein the leading edge (22) of the aerofoil-shaped baffle structure (14) faces the direction of flow of the fluid in the channel (8,8a,8b,8c).

8. The microfluidic chip (1,100) according to claim 1, wherein the channel (8,8a,8b,8c) has a formation selected from straight, a zig-zag, a circle, an oval, an ellipse, and any other curved shapes.

9. The microfluidic chip (1,100) according to claim 1, wherein the at least one channel (205,405) is a backflow-suppressing channel (205,405) positioned between, and in fluid communication with, the inlet (2,4,4b-4i) and the junction (5,5a,5b).

10. The microfluidic chip (1,100) according to claim 1, wherein the at least one channel (205,405) is a backflow-suppressing channel (205,405) positioned between, and in fluid communication with, the inlet (2,4,4b-4i) and the junction (5,5a,5b), and wherein the backflow-suppressing channel (205,405) comprises the non-aerofoil-shaped baffle structure (10) having a tilted rectangular baffle shape.

11. The microfluidic chip (1,100) according to claim 1, wherein the channel (8a) is a lipid mixing channel (8a) and the channel (8b) is a payload solution and aqueous solution mixing channel (8b) in fluid communication with the inlet (4,4a-4j) and the junction (5,5a,5b).

12. The microfluidic chip (1,100) according to claim 1, wherein the channel (8,8c) is a lipid nanoparticle synthesis channel (8,8c) in fluid communication with the junction (5) and the outlet (6).

13. The microfluidic chip (1,100) according to claim 1, wherein the channel (8,8c) is a lipid nanoparticle synthesis channel (8,8c) in fluid communication with the junction (5) and the outlet (6), and wherein the nanoparticle synthesising channel (8,8c) comprises the aerofoil-shaped baffle structure (14).

14. The microfluidic chip (1,100) according to claim 1, wherein the aerofoil-shaped baffle structure (14) has a shape selected from a flat-convex, a wing, a sail, and a blade of a propeller, a rotor, or a turbine.

15. The microfluidic chip (1,100) according to claim 1, wherein the channel (205,405,8,8a,8b,8c) and the microchannel (9) each have a cross-sectional shape selected from a rectangle, a square, a circle, an oval, and an ellipse.

16. The microfluidic chip (1,100) according to claim 1, wherein the width (16) of the channel (205,405, 8,8a,8b,8c) and the microchannel (9) is from about 50 μm to about 1000 μm, while the height (18) of the channel (205,405, 8,8a,8b,8c) and the microchannel (9) is from about 50 μm to about 500 μm.

17. The microfluidic chip (1,100) according to claim 1, wherein the fluid has a flow rate of between about 100 μl/min to about 20 ml/min.

18. (canceled)

19. A method for synthesising a nanoparticle, the method comprising the steps of:
(a) applying an organic solution to at least one inlet (4,4c-4h) of a microfluidic chip (100) comprising at least two inlets (2,4,4a-4j) in fluid communication with at least one channel (205,405) that meet at a junction (5,5a,5b) that is in fluid communication with a proximal end (3) of a channel (8,8a,8b,8c) thereof and an outlet (6) that is in fluid communication with a distal end (7) of the channel (8,8a,8b,8c), wherein the channel (8,8a,8b,8c) comprise a plurality of spaced-apart non-aerofoil-shaped baffle structures (10) continuous with an outer wall (11) of the channel (8,8a,8b,8c) and at least one aerofoil-shaped baffle structure (14) within the channel (8,8a,8b,8c), and wherein a microchannel (9) is formed around the spaced-apart non-aerofoil-shaped baffle structures (10) and the at least one aerofoil-shaped baffle structure (14) in the channel (8,8a,8b,8c) adapted to accommodate a fluid;
(b) applying an aqueous solution to at least one inlet (2,4a,4b,4i,4j) of the microfluidic chip (100) that was not used in step (a);
(c) applying pressure to the inlets (2,4,4a-4j) to set a desired flow rate through the channel (205,405), and thus (8,8a,8b,8c) and a flow rate ratio between channel (205, 405) and a total flow rate through channel (8,8a,8b,8c); and
(d) retrieving the synthesised nanoparticle from the outlet 6 that is in fluid communication with the channel (8,8c).

20. The method according to claim 19, wherein the channel (205,405) is a backflow-suppressing channel and a channel for pressure drop regulation.

21. The method according to claim 19, wherein the channel (8,8c) is a nanoparticle synthesising channel.
